(19) United States
(12) Patent Application Publication
(10) Pub. No.: US 2015/0107600 A1
(43) Pub. Date: Apr. 23, 2015

(54) Medical Bite Blocks or Mouthpieces

(71) Applicant: GI Supply, Inc., Camp Hill, PA (US)

(72) Inventors: Frank Wilson Jackson, Mechanicsburg, PA (US); Frank Wilson Jackson, III, Mechanicsburg, PA (US); William Robert Harding, Bloomsburg, PA (US)

(21) Appl. No.: 14/319,778

(22) Filed: Jun. 30, 2014

Related U.S. Application Data
(60) Provisional application No. 61/894,405, filed on Oct. 22, 2013.
(60) Provisional application No. 61/934,941, filed on Feb. 3, 2014.

(51) Int. Cl. A61B 1/00

(52) U.S. Cl. CPC A61B 1/00131
USPC 128/845

(57) ABSTRACT

The disclosure relates to bite blocks for endoscopic and related medical procedures, specifically bite blocks that achieve and maintain patient mandibular advance.

This application claims priority from co-pending U.S. Application No. 61/894,405 for “Adjustable Medical Bite Blocks” filed Oct. 22, 2013 and U.S. Application No. 61/934,941 for “Medical Bite Blocks or Mouthpieces” filed Feb. 3, 2014, the disclosures of which are incorporated herein by reference.

### FIELD OF THE DISCLOSURE

The disclosure relates to the field of bite blocks that provide patient mandibular advance in endoscopic and like medical procedures.

### BACKGROUND OF THE DISCLOSURE

In gastro-intestinal endoscopic procedures, instruments such as endoscopes may be inserted through a patient's mouth and into the patient's body. Bite blocks allow a physician to perform procedures without patient interference from biting into the instrument.

Bite blocks may include a through aperture or airway channel positioned in the patient's open mouth. An instrument is then inserted through the aperture or channel, through the patient's pharynx airway opening and into the patient's esophagus, stomach or other areas of the intestinal tract.

U.S. Pat. No. 5,174,284 discloses a related bite block that includes an aperture or channel that allows an endoscopic instrument to be inserted through the bite block and into a patient's body.

Medical, dental and upper gastro-intestinal endoscopic procedures are typically uncomfortable, requiring administration of a general anesthetic agent, such as propofol or a sedative agent such as midazolam, to a patient.

The use of general anesthetic or sedative agents is associated with the partial collapse of the patient's upper airway posterior to the base of the tongue at the retroglossal pharyngeal airway, commonly referred to as the collapsible pharyngeal airway. Partial collapse of the retroglossal pharyngeal airway during anesthetization or sedation restricts air flow to the respiratory system and may lower oxygen levels in the blood.

Likewise, insertion of an endoscope or other instrument through the patient's retroglossal pharyngeal airway during endoscopic and like procedures can further obstruct the airway, restricting air flow and respiration to lower patient blood oxygen levels.

To enlarge the airway and prevent airway collapse and obstruction, an anesthesiologist, doctor, nurse or like medical provider may manually position or advance the lower jaw or mandible in a forward position relative to the maxillary dentition of the patient to achieve “mandibular advance”. Mandibular advance increases the size of the patient's retroglossal pharyngeal airway and resists collapse of the airway.

Manual airway management by maintaining the mandible in the advance position during a medical procedure is difficult for medical providers. Continuous pressure on both sides of the posterior portion of the mandible must be maintained, occupying one or two hands of the medical provider throughout the procedure.

Bite blocks and related oral airway assemblies are known in the art to assist medical providers with airway management in endoscopic and like medical procedures.

Matioc, U.S. Pat. No. 8,640,692 and Flam, U.S. Pat. No. 5,590,643 disclose bite blocks having a block body and through aperture with a series of shallow ridges that extend across bottom surfaces of the bite block body. Each ridge has a front surface that extends away from the bite block body, but the front surface continuously and sharply curves to become parallel with the bottom surface of the bite block body while still relatively close to the bite block body. The shallow ridges define sets of shallow grooves along the bottom surface of the bite block body. During insertion into a mouth, the patient's mandible is manually adjusted forward by a medical professional so that a patient's lower front incisor teeth engage a selected shallow groove to achieve mandibular advance.

The Matioc and Flam bite blocks have disadvantages. The disclosed shallow grooves do not securely locate the patient's jaw during bite block procedures. In use, patient incisors must be placed into a precise groove for desired mandibular advance. Patient incisors become easily dislodged from a given shallow groove and not provide secure location of patient incisors. The curved forward surfaces of the ridges of the Matioc bite block enable the lower teeth to easily slide over the tops of the ridges if dislodged. Additionally, while falling under, during or awaking from the effect of anesthetics, the patient may shift mouth position and inadvertently place incisors in an undesired groove or involuntarily lose the motor control required to maintain incisor position within a specific groove. It is difficult to observe this and ongoing attention is required by a medical provider to assure that incisor position is maintained within a desired groove throughout the procedure. Further, the shallow grooves of the Matioc and Flam bite blocks are ineffective in maintaining mandibular advance in patients having misaligned or missing front incisors.

The Matioc and Flam bite blocks are additionally disadvantageous when patients are placed under an anesthetic or sedative. As an initial reaction to application of an anesthetic or sedative, patient jaw muscles become rigid and stiff, seizing the jaw in a locked position. This complicates installation of a bite block prior to or during the initial sedation. If a patient's lower incisors are not properly located in a desired groove, repositioning the block during this stage of a procedure is not possible.

Additionally, as application of an anesthetic or sedative continues, patient jaw muscles relax so that the jaw slacks into an open position in which a patient's lower incisors can easily become disengaged from a shallow locating groove thus losing desired mandibular advance.

To maintain a relatively open retroglossal pharyngeal airway, the Matioc and Flam bite blocks include a curved tongue retractor that extends downwardly into the patient's throat toward the upper esophagus during block installation. The tongue retractor maintains the patient's tongue in a forward position during a procedure to prevent the tongue from blocking the patient's upper airway when the patient's jaw relaxes with use of an anesthetic or sedative.

Use of a tongue retractor is highly undesirable as it is very uncomfortable for patients during installation, provoking patient gag reflex, causing the patient to shift position of the block undesirably, thus complicating block installation.

In the above cases, the installing medical provider must continually check the position of the block within the patient's mouth and/or maintain one or more hands on the patient through sedation or anesthetization and the medical procedure to assure that the patient's jaw is held at a desired location.

Therefore there is a need for a bite block achieving patient mandibular advance to achieve an open retroglossal pharyngeal airway throughout an endoscopic or other medical procedure that is easy to install by a medical professional and that securely maintains patient mandibular advance throughout medical procedures without the need for continued attention from the installing medical provider and does not require use of a tongue retractor portion to maintain an open airway.

### BRIEF SUMMARY OF THE DISCLOSURE

Disclosed is a bite block having mandible positioning features that achieve patient mandibular advance throughout endoscopic or like medical procedures. The bite block is easy to install by a medical professional and securely maintains patient mandibular advance throughout a medical procedure.

The disclosed bite block may be used for endoscopic medical procedures, related other upper or lower gastro-intestinal procedures, or other outpatient and ambulatory procedures.

The bite block includes a single lower jaw engagement member for locating the patient's lower jaw in an advanced position relative to the upper jaw. The lower jaw engagement member may include a single tooth/gum engagement surface. This surface is capable of engaging either the patient's lower incisor teeth or front gums for easy mouth installation that insures mandibular advance without need to locate patient incisors into a specific shallow ridge. Moreover, the tooth/gum engagement surface allows installation of the block in patients having misaligned or missing teeth or front incisors.

The lower jaw engagement member may include an engagement wall suitably sized to securely engage the patient's lower jaw without the need for continued attention from the installing medical provider and without the need for a tongue retractor to maintain proper block installation and an open patient airway.

In certain embodiments, the bite block may include a mouth roof palate engagement member that engages the patient's roof palate to stabilize the block during mouth installation. In other embodiments, the bite block may include a tooth engagement plate that engages the patient's molar teeth or back gums to stabilize the block during mouth installation.

Use of the disclosed bite block eliminates the need for a medical professional to manually maintain the mandible in a mandibular advance position during bite block installation and throughout the medical procedure.

Other objects and features of the disclosure will become apparent as the description proceeds, especially when taken in conjunction with the accompanying drawing sheets illustrating non-limiting embodiments.

### BRIEF DESCRIPTION OF THE DRAWINGS

FIG. 1 is an upper rear perspective view of first embodiment bite block;

FIG. 2 is a side view of the bite block of FIG. 1;

FIG. 3 is a top view of the bite block of FIG. 1;

FIG. 4 is a bottom of the bite block of FIG. 1;

FIG. 5 is front view of the bite block of FIG. 1;

FIG. 6 is a rear view of the bite block of FIG. 1;

FIG. 7 is a sectional view of the bite block taken along line 7-7 of FIG. 6;

FIG. 8 is a sectional view like FIG. 7 of a bite block showing an alternate embodiment lower jaw engagement member;

FIG. 9 is a sectional view like FIG. 7 of a bite block showing another alternate embodiment lower jaw engagement member;

FIG. 10 is a sectional view like FIG. 7 of a bite block showing another alternate embodiment lower jaw engagement member;

FIG. 11 is a sectional view like FIG. 7 of a bite block showing another alternate embodiment lower jaw engagement member;

FIG. 12 is an upper rear perspective view of an alternate embodiment bite block;

FIG. 13 is a side view of the bite block of FIG. 12;

FIG. 14 is a top view of the bite block of FIG. 12;

FIG. 15 is a bottom of the bite block of FIG. 12;

FIG. 16 is front view of the bite block of FIG. 12;

FIG. 17 is a rear view of the bite block of FIG. 12;

FIG. 18 is a sectional view of the bite block taken along line 18-18 of FIG. 17;

FIG. 19 is an upper rear perspective view of another alternate embodiment bite block;

FIG. 20 is a side view of the bite block of FIG. 15;

FIG. 21 is a top view of the bite block of FIG. 15;

FIG. 22 is a bottom of the bite block of FIG. 15;

FIG. 23 is front view of the bite block of FIG. 15;

FIG. 24 is a rear view of the bite block of FIG. 15;

FIG. 25 is a sectional view of the bite block taken along line 25-25 of FIG. 24;

FIG. 26 is a section view of the bite block taken along line 26-26 of FIG. 24;

FIG. 27 shows the bite block of FIG. 1 being installed into a patient's mouth;

FIG. 28 shows the bite block of FIG. 12 being installed into a patient's mouth;

FIG. 29 shows the bite block of FIG. 1 installed within a patient's mouth; and

FIG. 30 shows the bite block of FIG. 12 installed within a patient's mouth.

### DETAILED DESCRIPTION

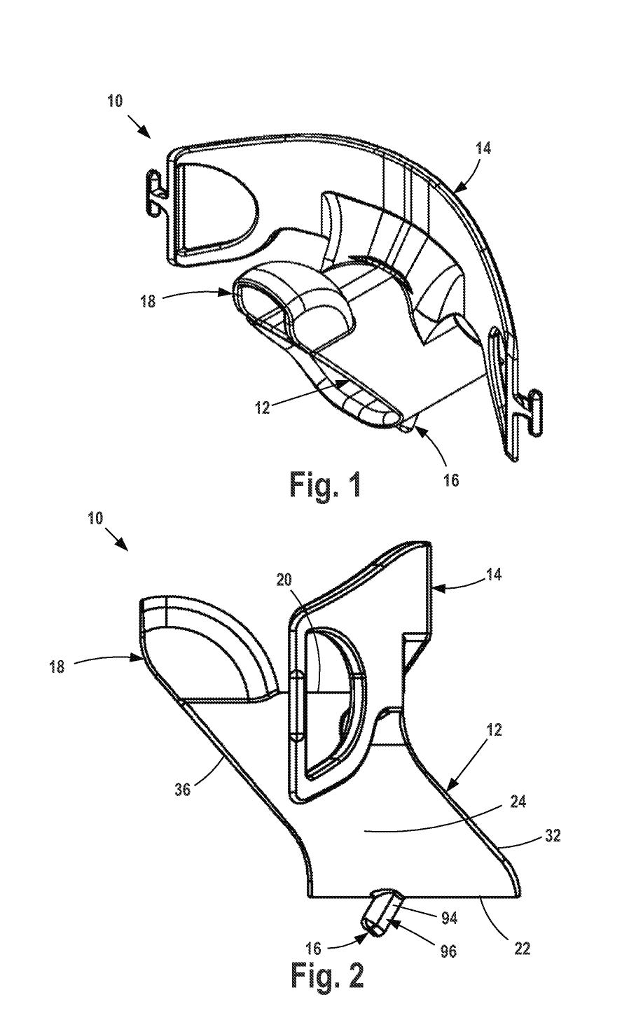

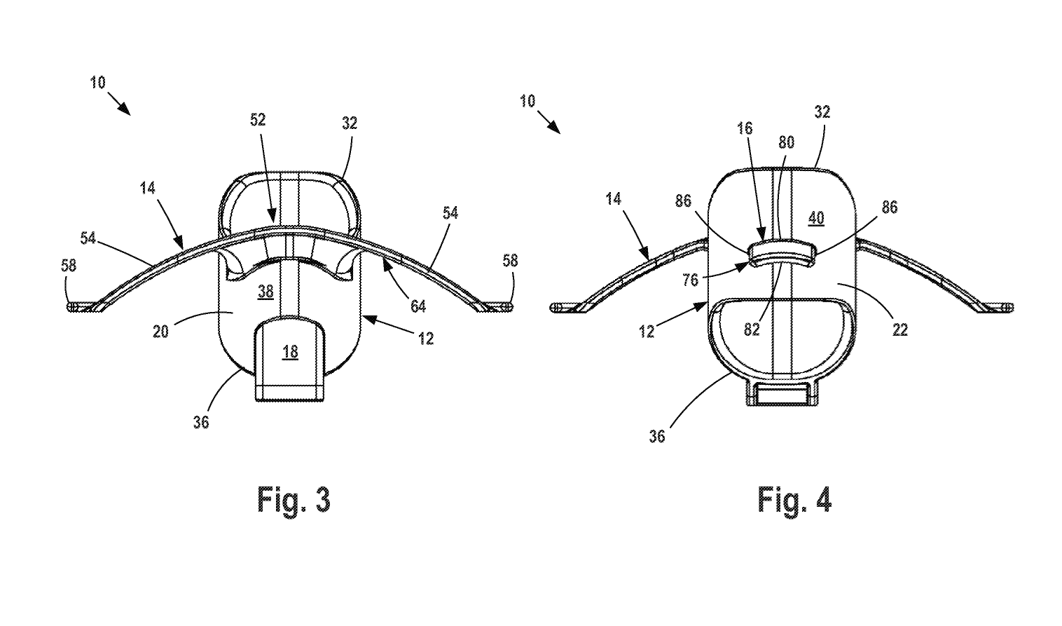

First embodiment endoscopic bite block 10 is illustrated in FIGS. 1 through 7 and may be formed as an integral, one-piece member from plastic or like material.

Bite block 10 has a block body 12, a front piece 14, lower jaw engagement member 16 and mouth roof palate engagement member 18.

Block body 12 is a tubular body that extends along a central longitudinal axis 28. The block body 12 has body top 20, body bottom 22 and body sides 24 and an interior channel 26 extending through the body along block central axis 28 from channel inlet 30 at body front end 32 to channel outlet 34 at body back end 36.

Body top surface 38 is located at block body top 20 and block body bottom surface 40 is located at body bottom 22.

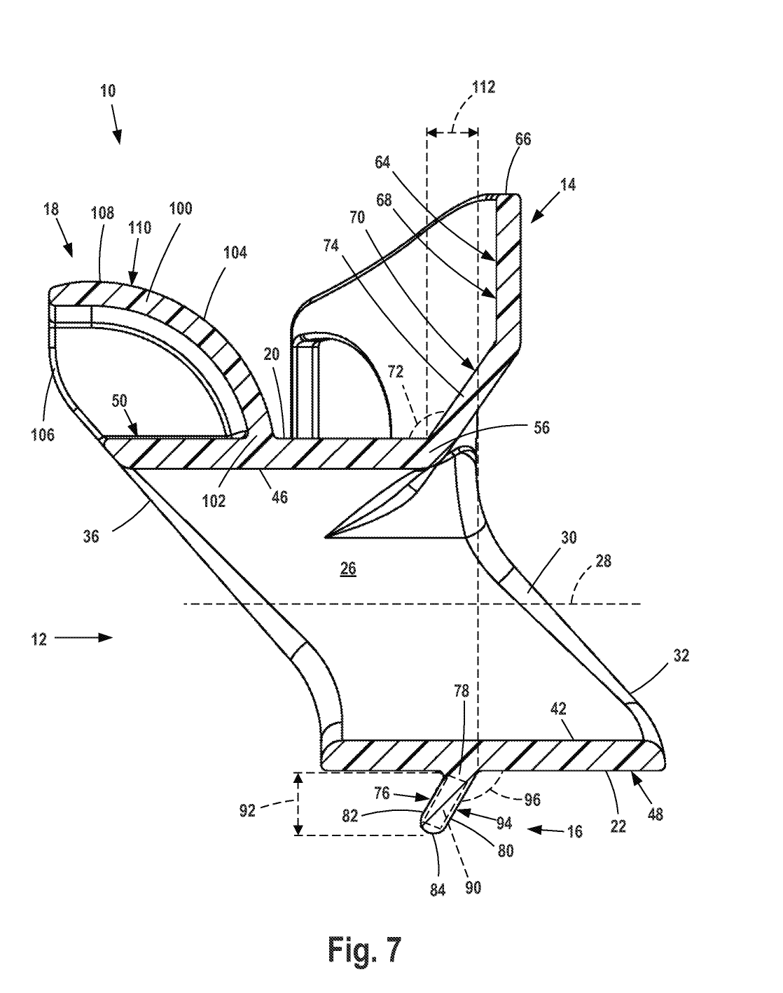

In embodiments, block body 10 may be uniform in cross section so that block body top surface 38 and block body bottom surface 40 extend generally parallel with the block central axis 28 and may be parallel with each other when viewed in cross-section. See FIG. 7.

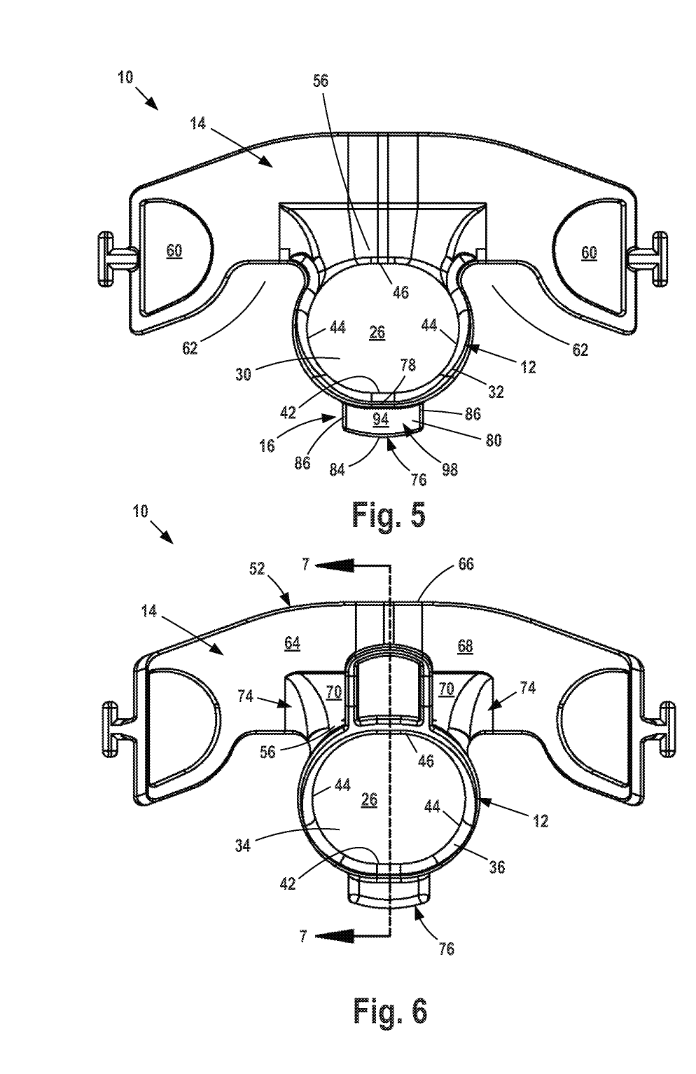

Channel 26 may be generally circular or oval in cross section and have a smooth interior surface extending from channel interior floor 42 upward to channel interior sides 44 and channel interior roof 46 as shown in FIGS. 5 and 6.

In embodiments wherein channel 26 has a generally uniform cross-section along the block central axis 28, the channel roof 42 and the channel floor 46 may extend parallel with each other as shown in FIG. 7.

Channel inlet 30 may be tapered upwardly and channel outlet 34 may be tapered downwardly by axially offsetting the body bottom 22 with respect to the body top 20 towards the body front 32 or body back 36. This enables the body bottom 22 to have under-hang portion 48 and the body top 20 to have over-hang portion 50.

Front piece 14 is adjacent to the front end of the block body 12 and is wider than the block body 12. Front piece 14 has a curved body 52 made up of integral front piece sub-pieces 54 joined to body top 20 proximate body front 32 at front piece base 56.

Anchors 58 are attached to the sub-pieces pieces 54 on opposite ends of front piece 14.

Each sub-member 54 may include a port 60 and/or cutaway portion 62 to allow additional patient mouth access during medical procedures.

Each sub-piece 54 includes concave interior surface 64 facing body back 36 and extending upward from front piece base 56 to front piece top 66. Interior surface 64 may have an upper jaw/lip engagement surface 68 extending upwardly from front piece base 56 toward front piece top 66. Upper jaw/lip engagement surface 68 may be generally continuous with interior surfaces 64.

Front piece base 56 may include contact surface 70 oriented at an angle 72 relative to block body top 20. Contact surface 70 may conform generally to front piece member curved body 52 so that contact surface 70 is a generally inwardly-curving frustoconical surface 74. As best seen in FIG. 7, angle 72 may be obtuse and greater than 90 degrees. In embodiments, angle 72 may be approximately 120 degrees.

Lower jaw engagement member 16 may be the sole projection or discontinuity present along the otherwise smooth and continuous block body bottom surface 40. In embodiments, there may be no other discontinuities, ridges, slots, or grooves formed in or on the block body bottom surface 40 between the lower jaw engagement member 16 and the front end 32 of the block body 12. The lower jaw engagement member 16 has a body 76 joined to block body bottom 22 at lower jaw engagement member body base 78. The body 76 is formed as a wall extending away from the block body 12 generally transverse to the central block axis 28.

Lower jaw engagement member body or wall 76 has a member body front surface or front wall surface 80 facing block body front 32 and member body back surface or back wall surface 82 facing block body back 36. Member body or wall 76 extends downwardly from member body or wall base 78 to member body end 84 and laterally to member body sides 86. Each of the front and back wall surfaces 80, 82 extend substantially the entire distance from the block body 20 to the wall free end 84.

As best seen in FIG. 7, the front and back wall surfaces 80, 82 extend parallel with each other from the wall base 78 to wall free end 84. The wall surfaces 80, 82 each extend at a constant slope with respect to the central block axis 28 from the block body 12 to the wall free end 84. Engagement wall 76 may have a generally uniform wall thickness extending from the member body front wall surface 80 to member body back wall surface 82. In embodiments, wall thickness may be about 3 millimeters.

Wall 76 may have generally uniform cross-sectional area 90 extending between member wall or body sides 86. As shown in FIG. 7, cross-sectional area 90 may be generally rectangular or parallelogram in shape.

Engagement body or wall 76 extends downwardly away from the block body 12 a lower jaw member body distance 92 from member body base 78 to member body end 84. In embodiments, distance 92 may be about 5 millimeters. In other embodiments, distance 92 may be more or less than 5 millimeters.

Engagement wall 76 may extend along a curved or arcuate path between the opposite wall sides 86. The cross section of wall 76 may be uniform along the wall path as shown in FIG. 7. The front wall surface 80 may be a convex surface and the back wall surface 82 may be a concave surface. The front wall surface 80 may be curved generally similarly to front piece body 52, so that body front wall surface 80 is convex and outwardly curved. Body front wall surface 80 may have a lower tooth/gum engagement surface 94 extending downwardly from proximate body base 78 to proximate member body end 84.

Member body lower tooth/gum engagement surface 94 may be a ramp surface to engage a patient's teeth and assist in proper orientation and/or rotation of the block within a patient's mouth as explained in greater detail below.

In embodiments, lower tooth/gum engagement surface 94 may be oriented at an angle 96 relative to block body bottom 22 and conform generally to curved member body 76 so that so that lower tooth/gum engagement surface 94 is a generally outwardly-curving frustoconical surface 98. As best shown in FIG. 7, angle 96 may be obtuse and greater than 90 degrees as measured from the block body bottom 22 adjacent the front wall surface 80 towards the front wall surface 80. In embodiments, angle 96 may be approximately 120 degrees.

Mouth roof palate engagement member 18 has a body 100 joined to block body top 20 proximate body back 36 at palate engagement member base 102. Mouth roof palate engagement member 18 may engage the roof of a patient's mouth to act as an anti-rotation member during block mouth installation.

Mouth roof palate engagement member body 100 has a body front 104 facing block body front 32 and member body back 106 facing block body back 36. Body 100 extends upwardly from member base 102 to member top 108. Member body 100 may include a top mouth roof palate engagement surface 110 extending along body front 104 and extending from member top 108 to member base 102. In embodiments, surface 110 may curve outwardly as shown in the Figures.

In embodiments, top mouth roof palate engagement surface 110 may be pliable or deformable. This may be accomplished by over-molding a pliable or deformable substance onto surface 110 composed of a polymer, elastomer, rubber or like flexible substance. Alternatively, the pliable structure may include a gel contained within a flexible pad. Use of a pliable or deformable surface may allow more comfortable engagement with a patient's mouth roof hard palate.

Lower tooth/gum engagement surface 94 is located a distance 112 forward or front of jaw/lip engagement surface 68 as measured along axis 28. Distance 112 relates to the mandibular advance distance achieved in a patient during block installation as described in greater detail below. In specific embodiments, distance 112 may be in the range of 3.35 to 4.35 millimeters.

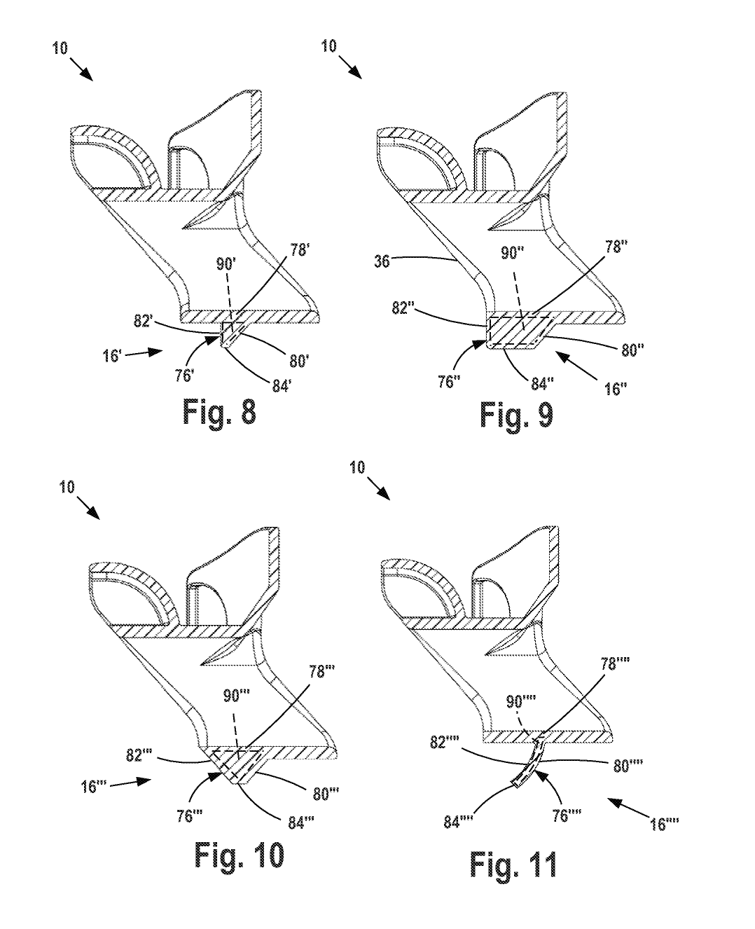

FIGS. 8 through 11 illustrate bite blocks 10 having alternate embodiment lower jaw engagement members 16. In particular, FIGS. 8 through 11 illustrate lower jaw engagement members 16 having alternate embodiment lower jaw engagement member bodies or walls 76.

FIG. 8 illustrates a lower jaw engagement member 16′ having a body or wall 76′ with a front surface or front wall surface 80′ and a member body back surface or back wall surface 82′. Body or wall 76′ extends downwardly from wall base 78′ to member body end 84′. Wall surface 80′ extends at an angle or slope with respect to the central block axis and wall surface 82′ extends generally perpendicular to the central block axis. Cross-sectional area 90′ is non-uniform and is generally triangular in shape.

FIG. 9 illustrates a lower jaw engagement member 16′″ having a body or wall 76′″ with a front surface or front wall surface 80′″ and a member body back surface or back wall surface 82′″. Back wall surface 82′″ is located proximate and continuous with block body back 36. Body or wall 76′″ extends downwardly from wall base 78′ to member body end 84′″. Wall surface 80′″ extends at an angle or slope with respect to the central block axis and wall surface 82′″ extends generally perpendicular to the central block axis. Cross-sectional area 90′″ is non-uniform and is generally quadrilateral in shape.

FIG. 10 illustrates a lower jaw engagement member 16′″ having a body or wall 76′″ with a front surface or front wall surface 80′″ and a member body back surface or back wall surface 82′″. Body or wall 76′″ extends downwardly from wall base 78′″ to member body end 84′″. Surfaces 80′″ and 82′″ extend toward each other at an angle or slope with respect to the central block axis. Cross-sectional area 90′″ is non-uniform and may be generally triangular in shape.

FIG. 11 illustrates a lower jaw engagement member 16″″ having curved or arcuate front and back surfaces 80″″ and 82″″. Front and back surfaces 80″″ and 82″″ extend generally parallel with each other from the wall base 78″″ to member body end 84″″ so that engagement wall 76″″ has a generally uniform wall thickness. Cross-sectional area 90″″ has the shape of a curved rectangle.

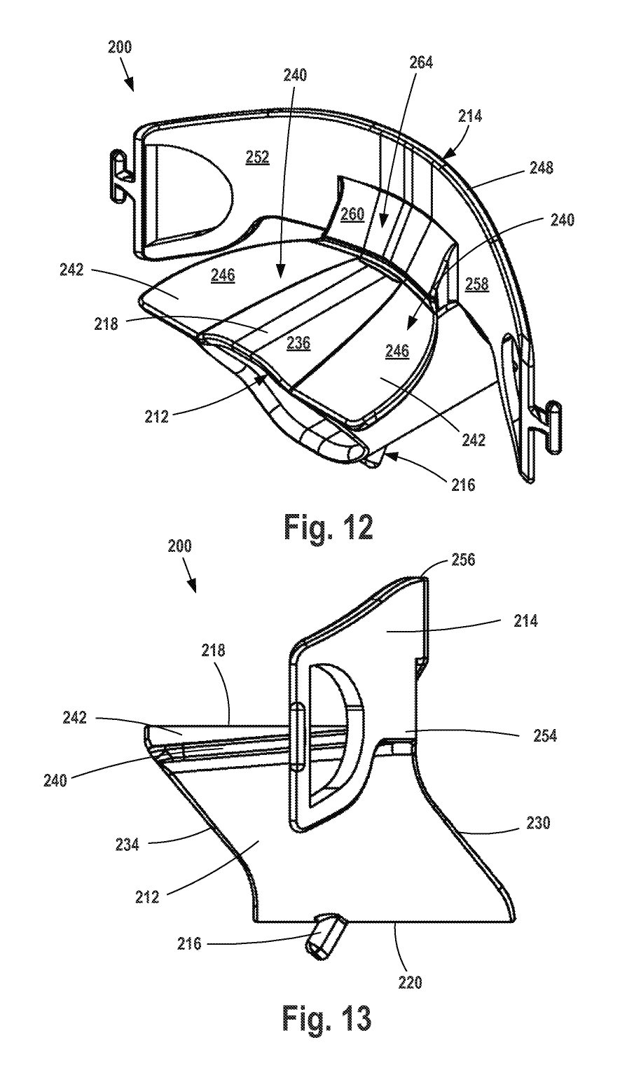

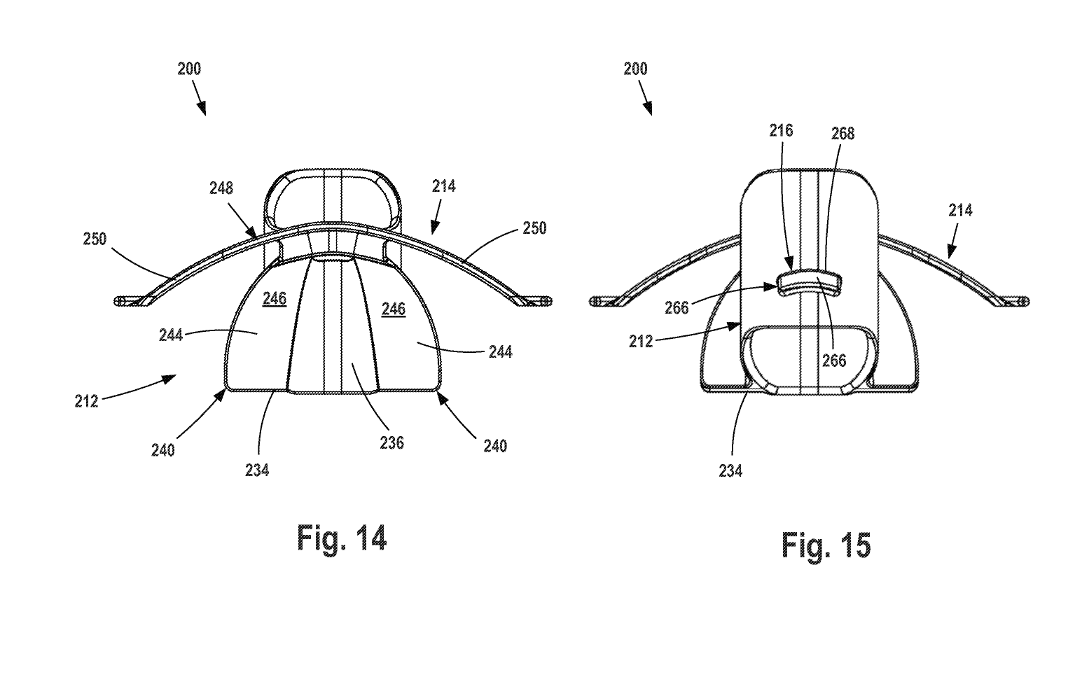

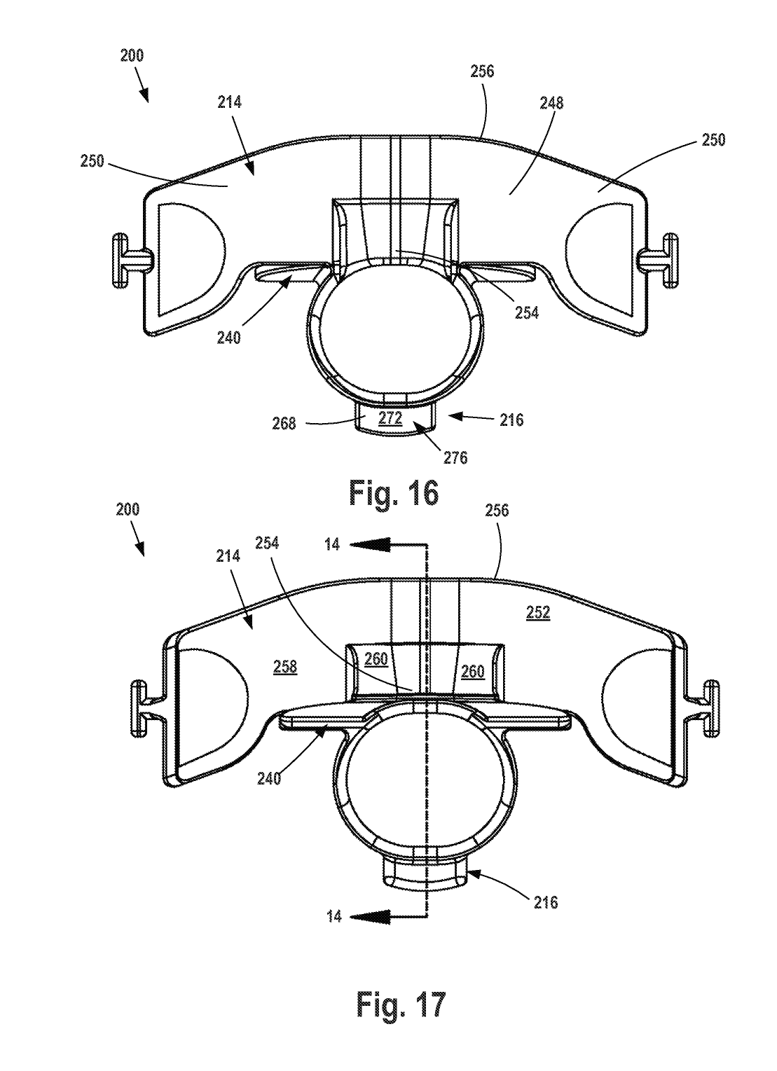

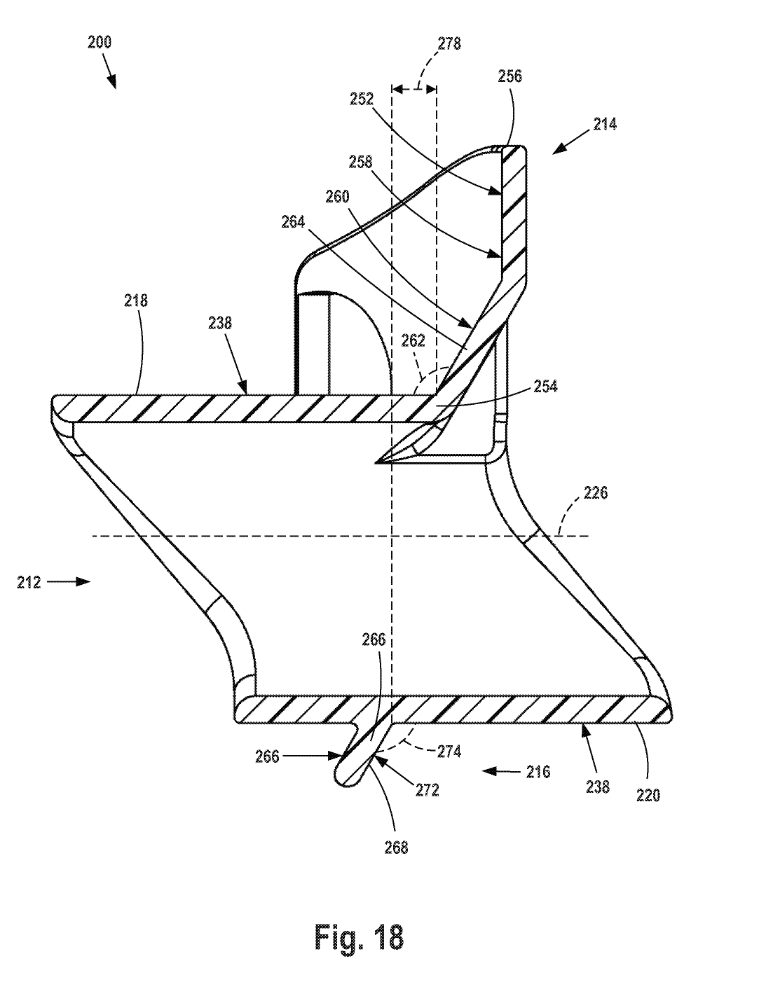

An alternate embodiment endoscopic bite block 200 is illustrated in FIGS. 12 through 18.

Bite block 200 is generally similar to and shares many elements with bite block 10. Bite block 200 may likewise be formed as an integral, one-piece member from plastic or like material.

Bite block 200 has a block body 212 similar to block body 12, a front piece 214 generally similar to front piece 14 and a lower jaw engagement member 216 similar to lower jaw engagement member 16.

Bite block body 212 has body top 218, body bottom 220, body sides 222 and a channel 224 like channel 26 described above. Channel 224 extends along block central axis 226 from channel inlet 228 at body front 230 to channel outlet 232 at body back 234.

Body top surface 236 is located at block body top 218 and block body bottom surface 238 is located at body bottom 220.

Bite block body 212 includes occlusal tooth engagement plate 240. Plate 240 is made up of plate members 242 integral with bite block body top 218 and extending above each body side 222. Plate 240 may have a generally uniform thickness as shown in the figures.

Each plate member 242 may have a member top surface 244 integral and continuous with body top surface 236. Member top surfaces 244 and body top surface 236 co-operate to form occlusal tooth engagement surface 246.

Occlusal tooth engagement surface 246 may be oriented generally co-planar with body top surface 236 or oriented at an angle relative to body top surface 236 as explained in greater detail in embodiments below.

Front piece 214 is similar to front piece 14 and has a curved body 248 made up of integral sub-pieces 250. Front piece 214 includes interior surface 252 facing block body back 234 and extending upward from front piece 254 to front piece top 256.

Interior surface 252 may have an upper jaw/lip engagement surface 258 like upper jaw/lip engagement surface 68 described above. Upper jaw/lip engagement surface 258 extends upwardly from front piece base 254 to front piece top 256 and may include contact surface 260 oriented at an angle 262 relative to block body top 236.

Additionally, contact surface 260 may conform generally to front piece curved body 248 so that contact surface 260 is a generally inwardly-curving frustoconical surface 264.

As shown in the figures, angle 262 may be obtuse and greater than 90 degrees. In embodiments, angle 262 may be approximately 120 degrees.

Lower jaw engagement member 216 is largely identical to lower jaw engagement member 16 disclosed above and having a member body or wall 266 like member body or wall 76 and having a member body front 268 like member body front 80. Member body front 268 may have a lower tooth/gum engagement surface 272 like lower tooth/gum engagement surface 94.

Lower tooth/gum engagement surface 272 may be oriented at an angle 274 relative to block body bottom 220 and conform generally to curved member body 226 so that lower tooth/gum engagement surface 232 is a generally outwardly-curving frustoconical surface 276. As shown in the figures, angle 234 may be obtuse and greater than 90 degrees. In embodiments, angle 274 may be approximately 120 degrees to allow comfortable engagement with a patient's teeth and/or gums.

Lower tooth/gum engagement surface 232 is located a distance 278 back or backward of upper jaw/lip engagement surface 258 as measured along block central axis 226. Distance 278 relates to the mandibular advance distance achieved in a patient during block installation as described in greater detail below. In certain embodiments, distance 278 may be about 4.2 millimeters.

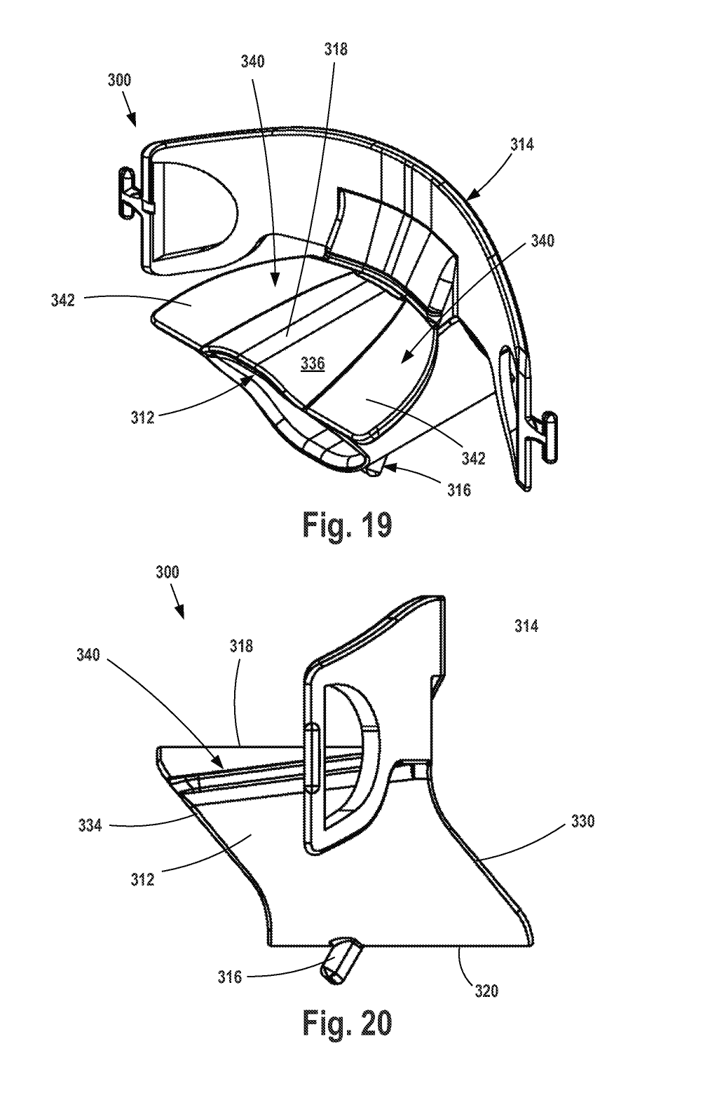

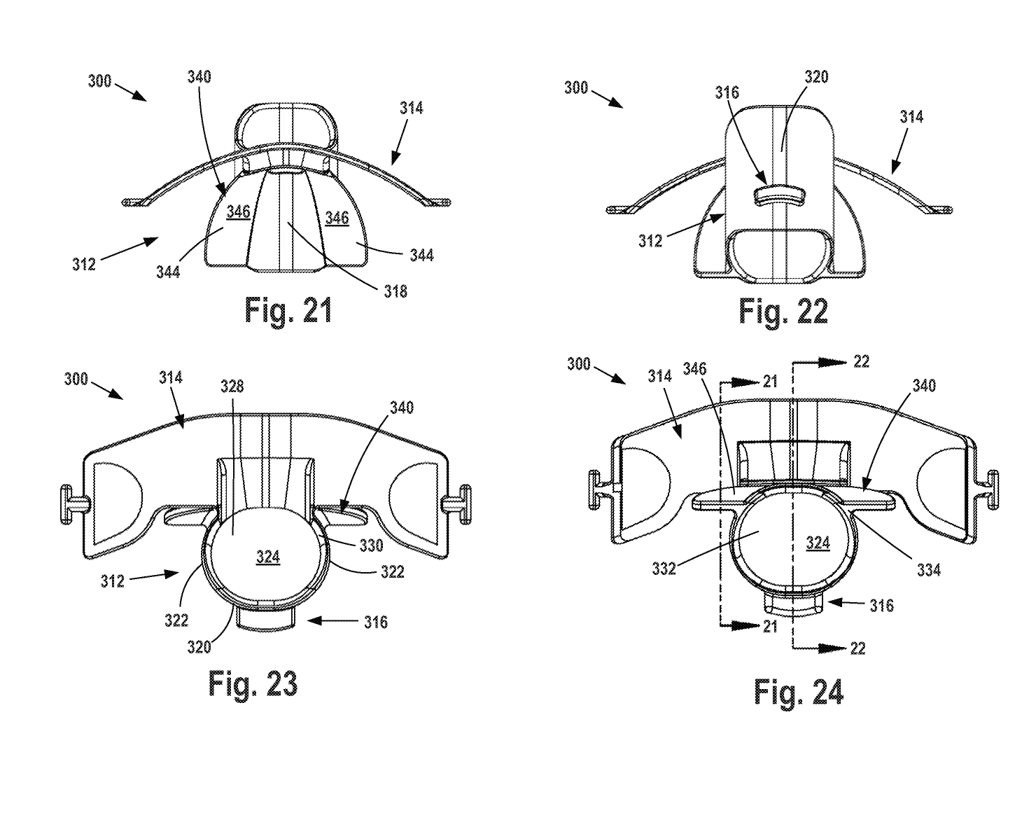

An alternate embodiment endoscopic bite block 300 is illustrated in FIGS. 19 through 26. Bite block 300 is generally similar to bite block 200.

Bite block 300 has a block body 312 similar to block body 212, a front piece 314 generally similar to front piece 214 and a lower jaw engagement member 316 similar to lower jaw engagement member 216.

Bite block body 312 has body top 318, body bottom 320, body sides 322 and a channel 324 like channel 224 described above. Channel 324 extends along block central axis 326 from channel inlet 328 at body front 330 to channel outlet 332 at body back 334.

Body top surface 336 is located at block body top 318 and block body bottom surface 338 is located at body bottom 320.

Bite block body 312 includes occlusal tooth engagement plate 340. Plate 340 is similar to plate 240 described above and is made up of two plate members 342 having member top surfaces 344. Top surfaces 344 cooperate to form occlusal tooth engagement surface 346 like occlusal tooth engagement surface 246.

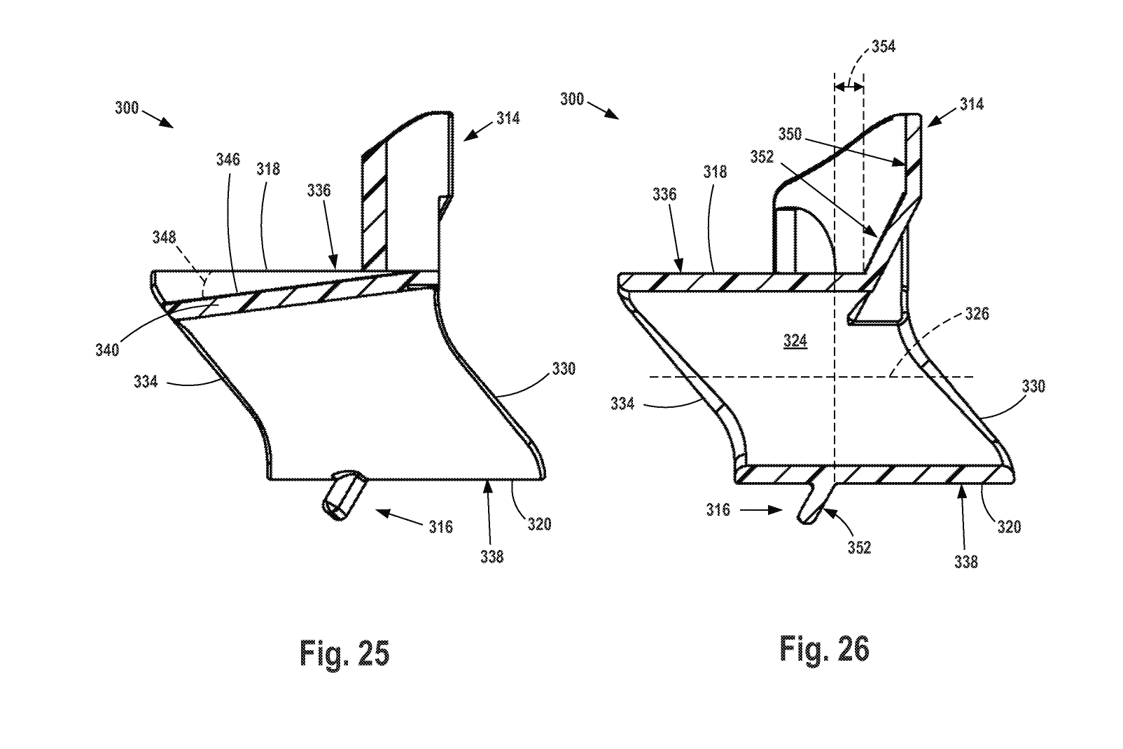

Plate 340 is oriented at an angle 348 downwardly of block body top 318 so that plate engagement surface 346 is likewise offset downwardly from body top surface 336 by angle 348. See FIG. 25. The orientation of plate 340 at angle 348 allows bite block 300 to be installed in a patient's mouth at an alternate angle. This allows more comfortable block installation in view of physical variations in patient mouths and allows an alternate orientation of block channel 324 relative to a patient's lower jaw line as explained in greater detail below. In certain embodiments, angle 348 may be about 7 degrees. In embodiments, plate 340 may be oriented at various angles relative to body top 318.

Bite block 300 front piece 314 may have an upper jaw/lip engagement surface 350 like upper jaw/lip engagement surface 258 disclosed above. Bite block 300 lower jaw engagement member 316 may have a lower tooth/gum engagement surface 352 like lower tooth/gum engagement surface 232 disclosed above.

Lower tooth/gum engagement surface 352 is located a distance 354 back or backward of upper jaw/lip engagement surface 350 as measured along block central axis 326. Distance 354 relates to the mandibular advance distance achieved in a patient during block installation as described in greater detail below. In certain embodiments, distance 354 may be about 4 millimeters.

Distances 106, 238 and 354 between the bite block upper and lower tooth/gum engagement surfaces may not necessarily directly correspond to the mandibular advance distance achieved in a patient once a bite block is installed in a patient's mouth. Bite blocks may rotate about block central axis at mouth installation to create a lower jaw mandibular advance distance different than distances 106, 238 and 354 as explained in greater detail below.

Use of bite blocks 10, 200 and 300 will now be described.

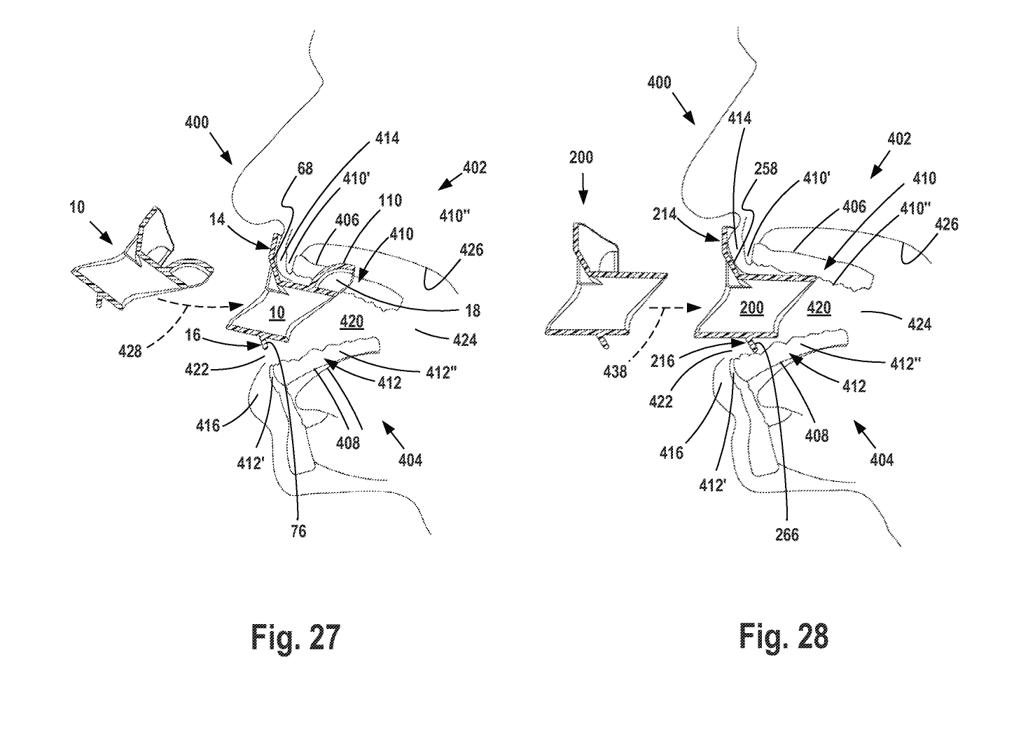

In use of bite block 10, a patient's mouth 400 is opened so that upper jaw 402 and lower jaw 404 are separated as shown in FIG. 27. Each jaw includes patient upper and lower gum line 406, 408 having upper and lower teeth 410, 412. Each jaw also includes upper and lower patient lips 414, 416. The patient's upper and lower teeth 410, 412 are made up of front incisor and canine teeth 410′ and 412′ and rear premolar and molar teeth 410″ and 412″. The patient's mouth includes oral cavity 420 located between jaws 402 and 404 and extending from oral cavity front 422 proximate lips 414, 416 to oral cavity back 424 proximate the patient's retroglossal pharyngeal airway at the back of the patient's throat (not-illustrated). Mouth roof palate 426 extends from proximate the patient's upper front incisor and canine teeth 410′ toward oral cavity back 424.

Bite block 10 may be inserted into patient mouth oral cavity 420 so that lower jaw engagement member 16 is located proximate lower jaw 404 and lower jaw engagement member body 76 is located proximate and/or behind lower gum line 408 and lower teeth 412. Simultaneously, front piece 14 is located proximate upper jaw 402 and upper jaw/lip engagement surface 68 is located in front of upper gum line 406 and upper teeth 410.

Sub-members 54 and the contact surfaces 70 co-operate to extend around the front upper lips and upper jaw of the patient when block 10 is placed in a patient's mouth and locate the block 10 axially with respect to the patient's upper jaw. The inclination of the contact surface 70 from vertical allows comfortable engagement of the surface 70 against the patient's upper jaw, lips, teeth and/or gums.

Bite block 10 may be positioned and/or rotated as it is inserted into patient mouth 400 along arrow 428 to properly locate upper and lower jaw engagement members 14, 16 relative to upper and lower jaws 402, 404 as indicated in FIG. 27.

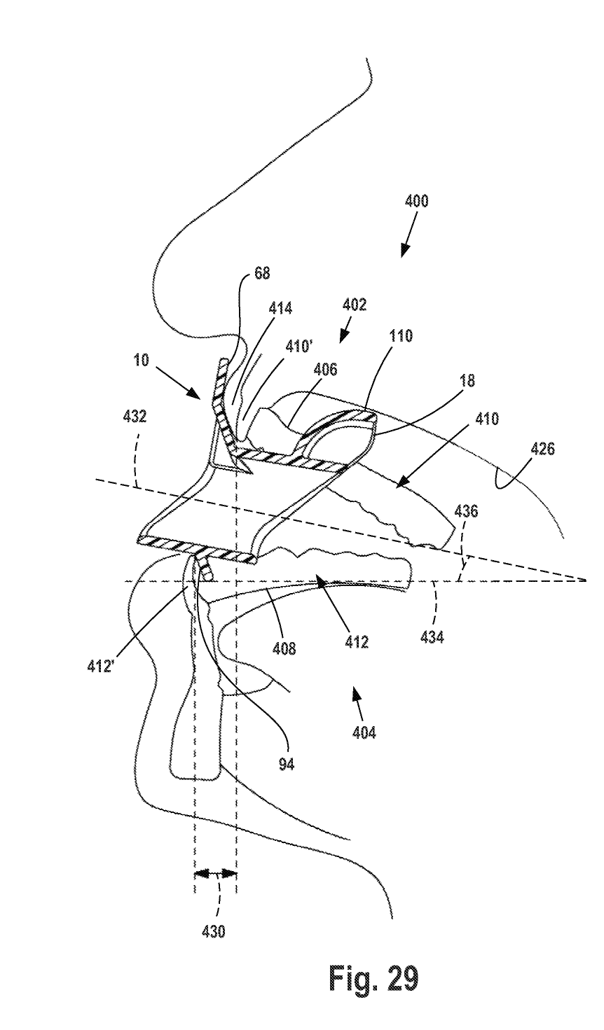

Next, the patient's mouth is closed so that upper jaw 402 and lower jaw 404 are brought together as shown in FIG. 29. As mouth 400 closes, jaws 402 and 404 engage and exert forces on bite block 10 to position and/or rotate the bite block into an installation position. In patients having front lower teeth 412′, patient teeth 412′ begin to engage lower tooth/gum engagement surface 94. Likewise, in patients lacking front lower teeth 412′, patient lower gum line 408 begins to engage lower tooth/gum engagement surface 94. Simultaneously, patient upper lip 414 begins to engage upper jaw/lip engagement surface 68.

As the patient mouth closes into the bite block installation position, depending on the specific positioning of block 10 within patient mouth 400, patient's lower teeth 412′ and/or lower gums 408 may either engage lower tooth/gum engagement surface 94 or a location on block body bottom surface 40 between the lower jaw engagement member 16 and the front end 32 of the block body 12.

In cases where patient lower teeth 412′ and/or lower gums 408 engage lower tooth/gum engagement surface 94 ramp surface, block 10 is positioned and/or rotated within patient mouth 400 as the teeth 412′ and/or gums 408 travel along engagement surface 94 to achieve a block installation position shown in FIG. 24.

In cases where patient lower teeth 412′ and/or lower gums 408 engage a location on block body bottom surface 40 between the lower jaw engagement member 16 and the front end 32 of the block body 12, block 10 is positioned and/or rotated within patient mouth 400 as the teeth 412′ and/or gums 408 travel along block body bottom surface 40 to achieve a block installation position shown in FIG. 29.

The distance the lower engagement wall 78 extends downwardly from the block body 12 and having the entire front wall surface 82 being transverse to the block axis 28 allows the front wall surface 82 to securely engage a patient's lower teeth and/or jawline during block installation. The extension distance of the lower engagement wall 78 can vary in other embodiments depending on the specific requirements for a block to properly engage a patient's teeth and/or jawline during block installation due to physical variation in different patient mouths.

Simultaneously, the patient's upper lip 414 engages upper jaw/lip engagement surface 70 so that patient upper lip 414 is sandwiched between upper jaw/lip engagement surface 70 and the teeth and/or gums of upper jaw 402. In the installation position, bite block 10 positions and/or rotates into an engagement position as shown in FIG. 29. Block positioning and/or rotation may halt when mouth roof palate engagement member 18 top mouth roof palate engagement surface 104 engages mouth roof palate 426.

Mouth roof palate 426 engagement by mouth roof palate engagement member 18 maintains stability of block 10 within the patient's mouth and also acts to resist rotation or dislodgement of block 10 as medical instruments are inserted through channel 26.

As block positioning and/or rotation proceeds, lower tooth/gum engagement surface 94 exerts a force against lower jaw 404 to push it forward a mandibular advance distance 430 relative to upper jaw 402 to achieve a desired patient mandibular advance distance.

In the installation position, block central axis 28 is rotated to block installation axis 432 offset from lower jaw axis 434 extending along lower jaw 404 by an installation degree 436. As illustrated in FIG. 29, installation degree 436 may be about 11 degrees.

Once bite block 10 achieves the installation position within a patient's mouth, the block is secured to the patient by tightening a band or strap (not shown) attached to anchors 58. The band or strap will extend around a patient's head to hold the bite block in place in a fixed relationship with respect to a patient's upper jaw. In the installation position, block under-hang portion 48 prevents lower lip 416 from interfering with instruments inserted through channel 26.

Use of bite block 200 is generally similar to use of bite block 10.

First, the patient's mouth 400 is opened so that upper jaw 402 and lower jaw 404 are separated as shown in FIG. 28. Bite block 200 may be inserted into mouth oral cavity 420 so that lower jaw engagement member 216 is located proximate lower jaw 404 and lower jaw engagement member body 266 is located behind lower gum line 408 and lower teeth 412. Simultaneously, front piece 214 is located proximate upper jaw 402 and upper lip engagement surface 258 is located in front of upper lip 414. In locating front piece 214 proximate upper jaw, rounded upper lip engagement surface 258 may likewise be located proximate other patient features located close to upper lip 414 including the patient's cheeks and nasolabial folds.

Bite block 200 may be positioned and/or rotated as it is inserted into patient mouth 400 along arrow 438 to properly locate upper and lower jaw engagement members 214, 216 relative to upper and lower jaws 402, 404 as indicated in FIG. 28.

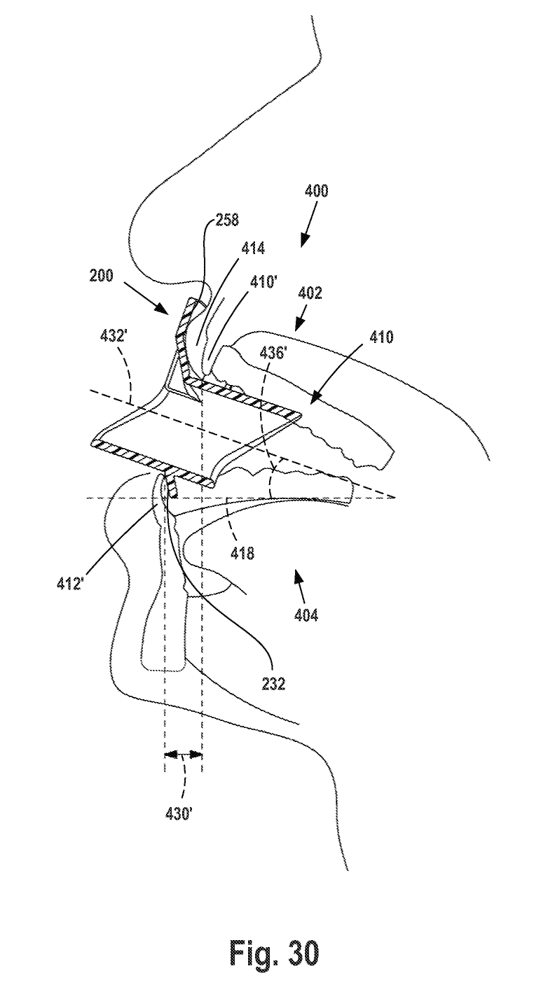

Next, the patient's mouth is closed so that upper jaw 402 and lower jaw 404 are brought together as shown in FIG. 30. As mouth 400 closes, jaws 402 and 404 engage and exert forces on bite block 200 to position and/or rotate the bite block into an installation position. In patients having front lower teeth 412′, patient teeth 412′ begin to engage lower tooth/gum engagement surface 232. Likewise, in patients lacking front lower teeth 412′, patient lower gum line 408 begins to engage lower tooth/gum engagement surface 232. Simultaneously, patient upper lip 414 and/or other patient features located close to upper lip 414 begin to engage upper lip engagement surface 258.

As the patient mouth closes into the bite block installation position, the patient's lower teeth 412′ and/or lower gums 408 fully engage the lower tooth/gum engagement surface 232, the patient's upper lip 414 and/or other patient features located close to upper lip 414 engage upper lip engagement surface 258 and bite block 200 positions and/or rotates into an installation position as shown in FIG. 30. Block positioning and/or rotation halts when patient upper rear premolar and molar teeth 410″ engage occlusal tooth engagement plate 240 occlusal tooth engagement surface 246.

Engagement of patient upper rear teeth 410″ on occlusal tooth engagement surface 246 maintains stability of the block 200 within the patient's mouth and also acts to resist rotation or dislodgement of block 200 as medical instruments are inserted through channel 224.

As block positioning and/or rotation proceeds, lower tooth/gum engagement surface 232 exerts a force against lower jaw 404 to push it forward a mandibular advance distance 430′ relative to upper jaw 402 to achieve a desired patient mandibular advance distance.

In the engagement position, block central axis 266 is rotated to block installation axis 432′ offset from lower jaw axis 418 by an installation degree 436′. As illustrated in FIG. 30, installation degree 436′ may be about 20 degrees.

Once bite block 200 is installed in a patient's mouth, the block is secured to the patient by tightening a band or strap attached to anchors on the front piece and about the patient's head to maintain the bite block installation position.

Use of bite block 300 is generally similar to use of bite block 200, but for the orientation of plate 340 at an angle 348 relative to block body top 318. As a patient's mouth closes and bite block 300 positions and/or rotates into the installation position, the patient's teeth and/or gums engage respective upper tooth/gum engagement surface 350 and lower tooth/gum engagement surface 352. Positioning and/or rotation halts when patient upper rear premolar and molar teeth 410″ engage occlusal tooth engagement plate 340 occlusal tooth engagement surface 346.

The orientation of plate 340 at an angle 348 changes the installation position of block 300 relative to the installation position block 200. This allows block 300 to be installed in a patient's mouth at an installation degree offset from the lower jaw axis 418 different from an installation degree 426′ achieved in use of block 200. This allows block 300 to be more comfortably installed by accounting for physical variations in patient mouths and allows an alternate orientations of block channel 324 relative to a patient's lower jaw axis 418 and mouth to facilitate insertion of medical instruments through channel 324 an into a patient's upper airway.

Once installed, the disclosed bite blocks achieve a desired mandibular advance distance in patients so that the patient's upper airway is opened for endoscopic and like medical procedures.

The bite blocks disclosed herein obtain a desired mandibular advance distance in patients. Mandibular advance distances may be in the range of about 4 mm to about 20 mm. In specific embodiments obtained mandibular advance distance may about 20 mm. Alternate embodiment bite blocks may have different distances 106 and 238 between the bite block upper and lower tooth/gum engagement surfaces and/or different installation degrees 426, 426′ within a patient's mouth to achieve a desired mandibular advance distance in patients.

The present application discloses bite blocks wherein distances 106 and 238 between bite block upper and lower tooth/gum engagement surfaces do not match the mandibular advance distance achieved in a patient once bite blocks are installed. This is due to bite block rotation relative to the lower jaw axis. It is understood that in alternate embodiments a bite block may be installed so that there is no bite block rotation and the distance between the bite block upper and lower tooth/gum engagement surfaces match the mandibular advance distance achieved in a patient at installation. In such embodiments the block channel axis and the block installation axis would be substantially co-linear.

Other embodiments of the bite blocks and mouthpieces disclosed herein may provide more or less mandibular advance. A medical practitioner selects a bite block or mouthpiece that provides sufficient mandibular advance during use to achieve therapeutic enlargement of the patient's retroglossal pharyngeal airway.

In further embodiments, the disclosed bite blocks and mouthpieces are contemplated to be part of a family of related bite blocks and mouthpieces, each configured to obtain a specific mandibular advance distance in a patient. In use, a medical provider may select one of the family bite blocks or mouthpieces depending on patient need to achieve a desired degree of retroglossal pharyngeal airway enlargement.

Use of the disclosed bite blocks is not limited only to endoscopic applications. In certain medical applications, the disclosed blocks may be used as airway assistance devices for maintaining patient airways during anesthetization or sedation.

### Features

Additional bite block embodiments may have the following non-limiting features, alone or in combination with other features, described as follows:

1. A bite block for providing mandibular advance when in use extending into a patient's mouth, the bite block comprising:

a tubular body having an interior channel extending through the body along an axis, the tubular body comprising a front end, a back end axially opposite the front end, a bottom surface extending between the front and back end, a top surface extending between the front and back end, a front end portion at the front of the body, and a back end portion extending from the front end portion to the back of the body, the top surface of the back end portion facing and engagable with the upper teeth or upper jaw of a patient when the bite block is in use, the bottom surface of the back end portion facing and engagable with the lower teeth or lower jaw of a patient when the bite block is in use;

a front piece attached to the front end portion of the tubular body, the front piece comprising a surface facing towards the back of the body, the front piece surface being capable of engaging the outside of the patient's mouth or lips and resisting further insertion of the bite block into the patient's mouth when the bite block is in use;

a rigid wall extending from the bottom surface of the back body portion away from the body to an upper end of the wall, the wall comprising a front wall surface facing the front end of the body, the front wall surface extending transverse to the body axis from the bottom surface of the back body to the upper end of the wall;

the front wall surface being engageable against a back side of the patient's lower teeth or lower gums when the bite block is in use, the front wall surface being axially spaced from the front piece wherein the front wall surface displaces the lower jaw from the normal relaxed position of the lower jaw towards the front of the body when engaging the back side of the patient's lower teeth or lower gums when the bite block is in use.

2. The bite block of feature 1 wherein the wall comprises a rear wall surface facing the back of the body, the rear wall surface being parallel with the front wall surface.

3. The bite block of feature 1 wherein the front wall surface extends not less than about 5 millimeters away from the bottom surface of the body measured in a direction perpendicular to the body axis.

4. The bite block of feature 1 wherein the bottom surface of the back portion of the bite block is a smooth surface from the wall to the front end portion of the bite block.

5. The bite block of feature 1 wherein the wall front surface and the bottom surface of the body adjacent the wall front surface define an obtuse angle.

6. The bite block of feature 6 wherein the obtuse angle is about 120 degrees.

7. The bite block of feature 1 wherein the surface of the front piece and the front wall surface are substantially parallel with one another.

8. The bite block of feature 1 wherein the wall includes opposite sides spaced apart in a direction transverse to the body axis, the wall extending along a curved path in the said transverse direction between the wall sides.

9. The bite block of feature 6 wherein the front wall surface is convex.

10. The bite block of feature 1 wherein the front wall surface is a convex surface inclined at an obtuse angle with respect to the body axis.

11. The bite block of feature 1 wherein the surface of the front piece and the front wall surface are axially spaced apart not less than 3.35 millimeters and not greater than 4.35 millimeters.

12. The bite block of feature 1 comprising an occlusal tooth engagement plate attached to the body and extending away from the body in a direction transverse to the body axis.

13. The bite block of feature 12 wherein the occlusal tooth engagement plate extends towards the back of the body and extends away from the upper side of the body as the plate extends towards the back of the body.

14. The bite block of feature 1 comprising an anti-rotation member extending from the top surface of the back portion of the body away from the body and being capable of abutting the roof of the mouth of the patient to resist movement of the back of the bite body towards the roof of the mouth during use of the bite block.

15. The bite block of feature 13 wherein the anti-rotation member comprises a pliable portion that abuts the roof of the mouth when limiting rotational movement of the bite block during use.

16. The bite block of feature 1 wherein the bite block is capable of generating a mandibular advance of not less than 4 millimeters and not more than 20 millimeters.

17. The bite block of feature 1 formed as an integral one-piece member.

18. A method for generating mandibular advance in a patient, the method comprising the steps of:

(a) providing a rigid and integral elongate body having a front end, an opposite back end, a back end portion at the back end, and a wall extending from the back end portion of the body away from the body to a free end of the wall, the wall having a front wall surface facing the front end of the body;

(b) inserting the back end of the body a predetermined distance from the front of the mouth and into the open mouth of the patient and thereby placing the back end portion of the body inside of the mouth, the wall being placed adjacent to the inner side of the lower teeth and/or lower gums of the patient;

(c) engaging the front wall surface of the wall against the inner side of the lower teeth and/or lower gums of the patient when the lower jaw of the patient is in a relaxed position; and

(d) after performing step (c), driving the front surface of the wall against the lower teeth and/or lower gums of the patient displacing the lower jaw towards the front of the mouth.

19. The method of feature 18 wherein step (c) comprises the steps of:

(e) having the patient close his or her mouth after insertion of the body inside the mouth of the patient;

(f) initially engaging the lower teeth and/or lower gums of the patient against the front wall surface while the patient is closing his or her mouth before the mouth is fully closed; and step (d) comprises the step of:

(g) having the patient continue to close his or her mouth after initial engagement of the lower teeth and/or lower gums with the front wall surface and clench the body between the jaws of the patient whereby the lower teeth and/or lower gums slide along the front wall surface towards the front of the body and the front wall surface displaces the lower jaw of the patient towards the front of the body as the lower teeth and/or lower gums slide along the front wall surface during the continued closing of the patient's mouth.

20. The method of feature 18 wherein step (c) comprises the step of:

(e) rotating the body and thereby engaging the front wall surface against the lower teeth and/or lower gums of the patient; and

step (d) comprises the step of:

(e) continuing the rotation of the body after the initial engagement of the front wall surface against the lower teeth and/or lower gums of the patient whereby continued rotation of the body causes the front wall surface to push the lower jaw of the patient in a mandibular advance direction.

21. The method of feature 18 further comprising the step of:

(e) rotating the body while performing step (b) to place the wall proximate to the lower teeth and/or lower gums of the patient.

22. The method of feature 18 wherein the lower jaw has a mandibular advance of not less than 4 millimeters and not more than 20 millimeters after performing step (e).

23. The method of feature 18 wherein the wall surface is a frustoconical surface.

24. An endoscopic bite block, comprising:

A block body having a body front, a body back, a body top surface, a body bottom surface and a channel extending from an inlet at the body front through the body to an outlet at the body back, the channel defining a channel axis extending along the channel;
a front piece extending upwardly from the body top surface and comprising an upper jaw/lip engagement surface facing the body back and extending upwardly from the body top surface;
a lower jaw engagement member extending downwardly from the block body bottom surface, the lower jaw engagement member comprising a member body having a member body base joined to the block body lower surface, a member body front, a member body back, member body sides, a member body thickness extending from the member body front to the member body back and a member body end located downwardly a lower jaw member body distance from the block body bottom surface, the member body front comprising a lower tooth/gum engagement surface facing the body front and located a first distance from the upper jaw/lip engagement surface as measured along the channel axis;
the block body further comprising an installation orientation wherein the body is rotated a degree so that the channel defines a channel installation axis extending generally along the channel from the inlet to the outlet and the lower tooth/gum tooth engagement surface is located a second distance from the upper jaw/lip engagement surface as measured along the channel installation axis.

25. The endoscopic bite block of feature 24 wherein the lower jaw engagement member body comprises an engagement wall.

26. The endoscopic bite block of feature 25 wherein the lower jaw member body distance is at least 5 millimeters.

27. The endoscopic bite block of feature 24 wherein the upper jaw/lip engagement surface is oriented at an angle relative to the block body upper surface and the lower tooth/gum engagement surface is oriented at an angle relative to the block body lower surface.

28. The endoscopic bite block of feature 27 wherein the upper tooth/gum engagement surface angle and the lower tooth/gum engagement surface angle are similar.

29. The endoscopic bite block of feature 28 wherein the upper tooth/gum engagement surface angle and the lower tooth/gum engagement surface angle are about 120 degrees.

30. The endoscopic bite block of feature 24 wherein the lower tooth/gum engagement surface is curved.

31. The endoscopic bite block of feature 29 wherein the lower tooth/gum engagement surface is frustoconical.

32. The endoscopic bite block of feature 25 wherein the engagement wall comprises an elongate body extending from one body side to another body side having a generally uniform body thickness.

33. The endoscopic bite block of feature 32 wherein the engagement wall comprises a uniform cross-sectional area.

34. The endoscopic bite block of feature 24 wherein the channel axis and the channel installation axis are substantially co-parallel and the first distance and second distance are substantially similar.

35. The endoscopic bite block of feature 24 wherein the channel installation axis is rotated an installation degree from the channel axis and the second distance is greater than the first distance.

36. The endoscopic bite block of feature 24 wherein the block body comprises a tooth engagement plate.

37. An endoscopic bite block, comprising a block body having a body front, a body back, body sides, body top and body bottom, the body further comprising a channel extending through the body from an inlet at the body front to an outlet at the body back, the channel defining a channel axis extending along the channel, an front piece extending upwardly from the body top and comprising an upper jaw/lip engagement surface, the upper jaw/lip engagement surface facing the body back and a lower jaw engagement member extending downwardly from the block body bottom, the lower jaw engagement member comprising a member body having a member body front, a member body back, member body sides, a member body thickness extending from the member body front to the member body back, a member body base joined to the block body lower surface and a member body end located a lower jaw engagement member distance downward from the block body lower surface, the member body front comprising a lower tooth/gum engagement surface extending from proximately the block body lower surface to proximately the body end, the lower tooth/gum engagement surface facing the body front and extending away from the body lower surface, the lower tooth/gum engagement surface located a first distance from the upper jaw/lip engagement surface as measured along the channel axis, the block body further comprising a block installation axis extending generally along the channel and whereby the lower tooth/gum engagement surface is located a second distance from the upper jaw/lip engagement surface as measured along the channel installation axis.

38. The endoscopic bite block of feature 37 wherein the lower jaw engagement member body has a generally uniform body thickness and a generally uniform cross-sectional area.

39. The endoscopic bite block of feature 37 wherein the lower jaw engagement member distance is greater than the member body thickness.

40. The endoscopic bite block of feature 38 wherein the lower jaw engagement member distance is at least 5 millimeters.

41. The endoscopic bite block of feature 38 wherein the lower jaw engagement member distance is approximately 5 millimeters and the member body thickness is approximately 3 millimeters.

42. The endoscopic bite block of feature 37 wherein the upper tooth/gum engagement surface angle and the lower tooth/gum engagement surface angle are about 120 degrees.

43. The endoscopic bite block of feature 42 wherein the upper or lower tooth/gum engagement surface is frustoconical.

44. The endoscopic bite block of feature 37 wherein the channel axis and the block installation axis are substantially co-linear and the first distance and second distance are substantially similar.

45. The endoscopic bite block of feature 37 wherein the block installation axis is different than the block channel axis.

46. The endoscopic bite block of feature 37 wherein the block body comprises a tooth engagement plate.

47. A method of obtaining patient mandibular advance comprising the steps of:

A. Providing a bite block having a block front and a front block, and a channel extending from the block front to the block back, a channel axis extending along the channel, a front piece extending upwardly from the block body and a lower jaw engagement member extending downwardly from the block body, the front piece comprising an upper jaw/lip engagement surface and the lower jaw engagement member comprising a lower tooth/gum engagement surface, the lower tooth/gum engagement surface located a first distance from the upper jaw/lip engagement surface as measured along the channel axis;

B. Providing a patient having a mouth comprising an upper jaw a lower jaw, each jaw having an upper and lower gum line and upper and lower lips, the patient further having a mouth roof palate an upper pharyngeal air space;

C. Separating the upper and lower jaws to open the patient's mouth;

D. Inserting the bite block into the patient's mouth so that lower jaw engagement member is proximate the patient lower jaw and the front piece is located proximate the patient upper jaw;

E. Closing the patient's upper and lower jaws to engage the bite block so that the upper jaw/lip engagement surface engages the patient upper lip and the lower tooth/gum engagement surface engages the lower gum line;

F. Positioning and/or rotating the bite block within the patient mouth as the patient upper and lower jaws close toward an engagement position so that the lower tooth/gum engagement surface exerts a force against the patient's lower jaw to push the lower jaw a mandibular advance distance forward of the upper jaw to open the patient's upper pharyngeal air space; and

G. Halting positioning of the bite block at the engagement position so that the lower jaw is located a desired mandibular advance distance forward of the upper jaw and the bite block has a block installation axis.

48. The method of feature 47 wherein the bite block of step G further comprises a block installation axis, the block installation axis offset from the channel axis.

49. The method of feature 48 wherein the mandibular advance distance is greater that the first distance.

50. The method of feature 47 wherein step A further comprises providing a bite block having a mouth roof palate engagement member and step G further comprises halting positioning of the bite block when the mouth roof palate engagement member engages the patient's mouth roof palate.

52. The method of feature 47 wherein step A further comprises providing a bite block having a tooth engagement plate and step G further comprises halting positioning of the bite block when the patient's jaw engages the tooth engagement plate.

While this disclosure includes one or more illustrative embodiments described in detail, it is understood that the one or more embodiments are each capable of modification and that the scope of this disclosure is not limited to the precise details set forth herein but include such modifications that would be obvious to a person of ordinary skill in the relevant art and fall within the purview of the following claims.

1. A bite block for providing mandibular advance when in use in a medical patient's mouth, the bite block comprising:
a tubular body having an interior channel extending through the body along an axis, the tubular body comprising a front end, a back end axially opposite the front end, top and bottom surfaces on opposed top and bottom sides of the body, facing and engagable with upper teeth or an upper jaw of the patient when the bite block is in use, the bottom surface facing and engagable with lower teeth or a lower jaw of the patient when the bite block is in use;
the body top and bottom surfaces defining a body plane extending through the axis, the body top surface and body bottom surface, the body plane defining a vertical direction extending through the top and bottom surface along the body plane and a horizontal direction extending perpendicular to the vertical direction;
a front piece attached to the front end of the tubular body, the front piece comprising a surface facing towards the back end of the body, the front piece surface being capable of engaging the outside of the patient's mouth or lips and resisting further insertion of the bite block into the patient's mouth when the bite block is in use;
a wall extending from the bottom surface of the body to an upper end of the wall, the wall comprising a front wall surface facing the front end of the body, the front wall surface extending proximate the bottom surface of the body toward the upper end of the wall;
at least one of a mouth roof palate engagement member or a tooth engagement plate;
the mouth roof palate engagement member, if present, attached to the body top surface and extending vertically away from the top surface of the body, the mouth roof palate engagement member comprising a mouth roof palate engagement surface spaced away from the body, the mouth roof palate engagement surface facing away from the wall and engagable with a mouth roof palate of a patient to resist rotation of the bite block when the bite block is in use;
the tooth engagement plate, if present, attached to the body and extending in a horizontal direction away from the body, the tooth engagement plate comprising a tooth engagement surface on one side of the plate and facing away from the wall, the tooth engagement surface engagable with upper teeth or a upper jaw of a patient to resist rotation of the bite block when the bite block is in use;
the front wall surface being engageable against a back side of the patient's lower teeth or gums of the lower jaw when the bite block is in use, the front wall surface being axially spaced from the front piece wherein the front wall surface displaces the lower jaw from the normal relaxed position of the lower jaw towards the front of the body when engaging the back side of the patient's lower teeth or lower gums when the bite block is in use wherein the mouth roof palate member or the tooth engagement plate resist block rotation when the bite block is in use.

2. The bite block of claim 1 wherein the tooth engagement surface is located between the body top surface and the body bottom surface.

3. The bite block of claim 1 wherein the channel comprises an inlet at the body front, the channel extending to an outlet at the body back, the outlet tapered downwardly and comprising an over-hang portion.

4. The bite block of claim 1 wherein the body is generally symmetrical about the body plane.

5. The bite block of claim 1 wherein the wall includes opposite sides spaced apart in the horizontal direction, the wall extending along a curved path in the horizontal direction between the wall sides.

6. The bite block of claim 1 wherein the front wall surface extends at an obtuse angle with respect to the bottom surface.

7. The bite block of claim 2 wherein the tooth engagement surface is oriented at an angle relative to the body top surface.

8. The bite block of claim 1 wherein the bite block is capable of generating a mandibular advance of not less than 4 millimeters and not more than 20 millimeters.

9. A method for generating mandibular advance in a patient, the method comprising the steps of:
(a) providing a rigid and integral elongate body having a front end, an opposite back end, a back end portion at the back end, and a wall extending from the back end portion of the body away from the body to a free end of the wall, the wall having a front wall surface facing the front end of the body;
(b) inserting the back end of the body a predetermined distance from the front of the mouth and into the open mouth of the patient and thereby placing the back end portion of the body inside of the mouth, the wall being placed adjacent to the inner side of the lower teeth and/or lower gums of the patient;
(c) engaging the front wall surface of the wall against the inner side of the lower teeth and/or lower gums of the patient when the lower jaw of the patient is in a relaxed position; and
(d) after performing step (c), driving the front surface of the wall against the lower teeth and/or lower gums of the patient displacing the lower jaw towards the front of the mouth.

10. The method of claim 9 wherein step (c) comprises the steps of:
(e) having the patient close his or her mouth after insertion of the body inside the mouth of the patient;
(f) initially engaging the lower teeth and/or lower gums of the patient against the front wall surface while the patient is closing his or her mouth before the mouth is fully closed; and step (d) comprises the step of:
(g) having the patient continue to close his or her mouth after initial engagement of the lower teeth and/or lower gums with the front wall surface and clench the body between the jaws of the patient whereby the lower teeth and/or lower gums slide along the front wall surface towards the front of the body and the front wall surface displaces the lower jaw of the patient towards the front of the body as the lower teeth and/or lower gums slide along the front wall surface during the continued closing of the patient's mouth.

11. The method of claim 9 wherein step (c) comprises the step of:
(e) rotating the body and thereby engaging the front wall surface against the lower teeth and/or lower gums of the patient; and step (d) comprises the step of:
(e) continuing the rotation of the body after the initial engagement of the front wall surface against the lower teeth and/or lower gums of the patient whereby continued rotation of the body causes the front wall surface to push the lower jaw of the patient in a mandibular advance direction.

12. The method of claim 9 further comprising the step of:
(e) rotating the body while performing step (b) to place the wall proximate to the lower teeth and/or lower gums of the patient.

13. An endoscopic bite block, comprising:
A block body having a body front, a body back, body top and bottom surfaces on opposed top and bottom sides of the body, and a channel extending from an inlet at the body front through the body to an outlet at the body back, the channel defining a channel axis extending along the channel;
a body plane extending through the axis, the body top surface and body bottom surface, the body plane defining a vertical direction extending along the body plane and a horizontal direction extending perpendicular to the body plane;
a front piece extending vertically upwardly from the body top surface and comprising an upper jaw/lip engagement surface facing the body back and extending away from the body top surface;
a lower jaw engagement member extending vertically downwardly from the block body bottom surface, the lower jaw engagement member comprising a member body joined to the block body bottom surface, a member body front, and a member body end located downwardly a lower jaw member body distance from the block body bottom surface, the member body front comprising a lower tooth/gum engagement surface facing the body front and located a first distance from the upper jaw/lip engagement surface as measured along the channel axis;
an tooth engagement plate extending in a horizontal direction away from the block body, the tooth engagement plate comprising a tooth engagement surface on one side of the plate and facing away from the lower jaw engagement member;
the block body further comprising an installation orientation wherein the body is rotated a degree so that the channel defines a channel installation axis extending generally along the channel from the inlet to the outlet and the lower tooth/gum tooth engagement surface is located a second distance from the upper jaw/lip engagement surface as measured along the channel installation axis.

14. The endoscopic bite block of claim 13 wherein the lower jaw member body distance is at least 5 millimeters.

15. The endoscopic bite block of claim 13 wherein the upper jaw/lip engagement surface is oriented at an angle relative to the block body upper surface and the lower tooth/gum engagement surface is oriented at an angle relative to the block body lower surface.

16. The endoscopic bite block of claim 15 wherein the upper tooth/gum engagement surface angle and the lower jaw/lip engagement surface angle are similar.

17. The endoscopic bite block of claim 13 wherein the lower tooth/gum engagement surface is curved between the member body sides.

18. The endoscopic bite block of claim 17 wherein the lower tooth/gum engagement surface is frustoconical.

19. The endoscopic bite block of claim 13 wherein the lower jaw engagement member body comprises a generally uniform body thickness.

20. The endoscopic bite block of claim 13 wherein the tooth engagement surface is co-planar with the body top surface.

21. The endoscopic bite block of claim 13 wherein the tooth engagement plate comprises two plate members, each member having a plate member top surface, the tooth engagement surface comprised at least in part by the plate member top surfaces.

22. The endoscopic bite block of claim 21 wherein the plate member top surfaces are co-planar with the body top surface.

23. The endoscopic bite block of claim 13 wherein the tooth engagement surface is oriented at an angle relative to the body top surface.

24. An endoscopic bite block, comprising a block body having a body front, a body back, top and bottom surfaces on opposed top and bottom sides of the body, the body further comprising a channel extending through the body from an inlet at the body front to an outlet at the body back, the channel defining a channel axis extending along the channel, an front piece extending upwardly from the body top and comprising an upper jaw/lip engagement surface, the upper jaw/lip engagement surface facing the body back and a lower jaw engagement member extending downwardly from the block body bottom and comprising a member body having a member body front, a member body back, member body sides, a member body thickness extending from the member body front to the member body back, a member body base joined to the block body bottom surface and a member body end located a lower jaw engagement member distance downward from the block body bottom surface, the member lower jaw engagement body front comprising a lower tooth/gum engagement surface extending from proximately the block body bottom surface to proximately the lower jaw engagement body end, the lower tooth/gum engagement surface facing the body front and extending away from the body bottom side, the lower tooth/gum engagement surface located a first distance from the upper jaw/lip engagement surface as measured along the channel axis, the block body comprising an tooth engagement plate extending from either block body side, the tooth engagement plate comprising an tooth engagement surface facing away from the lower jaw engagement member, the block body further comprising a block installation axis extending generally along the channel and whereby the lower tooth/gum engagement surface is located a second distance from the upper jaw/lip engagement surface as measured along the channel installation axis.

25. The endoscopic bite block of claim 24 wherein the lower jaw engagement member body has a generally uniform body thickness and a generally uniform cross-sectional area.

26. The endoscopic bite block of claim 24 wherein the lower jaw engagement member distance is greater than the member body thickness.

27. The endoscopic bite block of claim 26 wherein the lower jaw engagement member distance is at least 5 millimeters.

28. The endoscopic bite block of claim 26 wherein the lower jaw engagement member distance is approximately 5 millimeters and the member body thickness is approximately 3 millimeters.

29. The endoscopic bite block of feature 24 wherein the channel axis and the block installation axis are substantially co-linear and the first distance and second distance are substantially similar.

30. The endoscopic bite block of claim 24 wherein the tooth engagement surface is oriented at an angle relative to the channel axis.
